(19) United States
(12) Patent Application Publication
(10) Pub. No.: US 2003/0125760 A1
(43) Pub. Date: Jul. 3, 2003

(54) Hollow needle

(75) Inventors: Ulrich J. Pfeiffer, Munchen (DE); Thomas Thalmeier, Munchsmunster (DE)

Correspondence Address: NIXON PEABODY, LLP, 8180 GREENSBORO DRIVE, SUITE 800, MCLEAN, VA, 22102, US

(73) Assignee: Pulsion Medical Systems AG, Munchen (DE)

(21) Appl. No.: 10/218,089

(22) Filed: Aug. 14, 2002

Related U.S. Application Data
(63) Continuation-in-part of application No. 09/803,982, filed on Mar. 13, 2001, now abandoned.

(30) Foreign Application Priority Data
Oct. 31, 2000 (DE) 100 53 883.5

(51) Int. Cl. A61B 17/34

(52) U.S. Cl. 606/185; 604/272

(57) ABSTRACT

The outer diameter of a hollow needle, comprising a ground surface at its tip, expands continuously from a region near the tip but behind the ground surface such that the outer diameter in the region located further from the tip is approximately double. The rotationally symmetrical transition region is conical and rounded off at each end.

In use, the ground tip surface of the puncture needle cuts through the skin, the intermediate tissue and the wall of the blood vessel, the wider part of the hollow needle causes the puncture point to widen. Due to having a primary puncture of small diameter and then widening the puncture hole to the diameter required for introducing a correspondingly thick catheter, the traumatic injury to the blood vessel thereafter recedes relatively quickly back to the smaller diameter of the puncture. Thus, in contrast to the use of a uniformly thick needle having just one outer diameter, the time for healing the wound is limited to that required for a smaller hole.

### CROSS-REFERENCE TO RELATED APPLICATIONS

This application is a continuation-in-part of U.S. patent application Ser. No. 09/803,982.

### BACKGROUND OF THE INVENTION

1. Field of the Invention

The present invention relates to a hollow needle for puncturing blood vessels and for introducing a wire, especially for the placement of catheters or the like.

2. Description of Related Art

Hollow needles for puncturing blood vessels and for introducing a wire, commonly referred to as puncture needles or cannulas, are employed, inter alia, for the application of a catheter by means of the so-called Seldinger technique. This process generally proceeds as follows: The blood vessel, into which the catheter is to be placed, is punctured with the hollow needle. By means of the hollow needle there is introduce a guide wire, which remains in the blood vessel while the hollow needle is then withdrawn. Before the catheter is threaded onto the guide wire and is introduced into the blood vessel thereby, it is usually necessary to widen the puncture point by employing a so-called dilator. A dilator is a sleeve of plastics material which extends conically towards the one end and is guided to the puncture point by the guide wire in order to widen it out to match the outer diameter of the catheter.

U.S. Pat. No. 2,541,542 to Perez discloses an instrument for obtaining tissues samples from a body including a style and a cannula. The cannula comprises a major tubular body portion of substantially the same diameter throughout the length thereof. The base portion of the cannula is not part of the body portion in a sense that it rather functions as a socket for the style. Moreover, the base portion is not adapted to widen a puncture hole. On the contrary, the base portion is not even intended to be inserted through the puncture hole into a patient's body at all. The style, on the other hand, is a solid object and therefore is not a hollow needle.

U.S. Pat. No. 5,002,535 to Gross discloses a stepped needle for use in spinal anesthesia, which includes a first elongated tubular portion of a larger diameter extending from a proximal hollow hub, and a second elongated tubular end portion of a smaller diameter located distally from the first tubular portion. A thin plastic catheter may be threaded through the stepped needle into the subarachnoid space of a patient's spinal chord.

### SUMMARY OF THE INVENTION

Based upon the background of the general requirements in the medical field, that every operation on the human body should be carried out as carefully as possible and with a low level of risk, that they should not be drawn out unduly and that the number of avoidable steps involved in an operation be reduced correspondingly, the object of the invention is to produce a device which, in use, will speed up and simplify the placement of the catheter in the blood vessels without dispensing with the advantages associated with the use of a conventional dilator.

In accordance with the present invention, this object is achieved by a hollow needle for puncturing blood vessels and for introducing a wire, especially for the placement of catheters or the like, whereby the needle has a first outer diameter in a first region near its tip, and has a second outer diameter in a second region which is located further from the tip than the first region, said second outer diameter being greater than the first outer diameter, preferably being 1.2 to 3.0 times greater. Such a needle has the advantage that the puncturing and careful widening of the puncture point is done in one step. The advantages of a conventional dilator are thereby retained but without having to utilize such a dilator. By dispensing with the dilator, one less auxiliary means needs to be obtained, stored and sterilized, so that apart from the costs for the actual operation, the accompanying costs are also reduced.

Preferably, the needle has a ground surface in the vicinity of the tip.

In one advantageous embodiment of the present invention, the first outer diameter is between 0.4 and 1.5 mm.

Preferably, the cross section of the hollow needle expands continuously in a transition region from the first outer diameter to the second outer diameter. It is expedient if the transition region has a conical shape, whereby the opening or aperture angle between the longitudinal axis of the hollow needle and an envelope line lying in a common plane in the conical transition region preferably lies between 100 and 45°.

The transition region advantageously begins behind the ground surface.

It is also expedient if the transition between the first and the second outer diameter is rounded off.

In a particularly preferred embodiment of the present invention, the regions having the first and second outer diameters and the transition region are formed in one piece. The reduced expenditure in comparison with the use of a dilator as an additional help means is particularly noticeable hereby.

### BRIEF DESCRIPTION OF THE DRAWINGS

An embodiment of the present invention will be explained hereinafter with the help of the schematic drawings which are not drawn to scale and in which:

FIG. 1 shows a schematic view of the front part of a hollow needle in accordance with the invention.

FIG. 2 shows a longitudinal section through the front part of a hollow needle in accordance with the invention illustrated in FIG. 1, whereby the needle is rotated about its longitudinal axis by 90°.

FIG. 3 shows a schematic view of the front part of a hollow needle in accordance with a second embodiment of the invention.

### DESCRIPTION OF THE PREFERRED EMBODIMENT

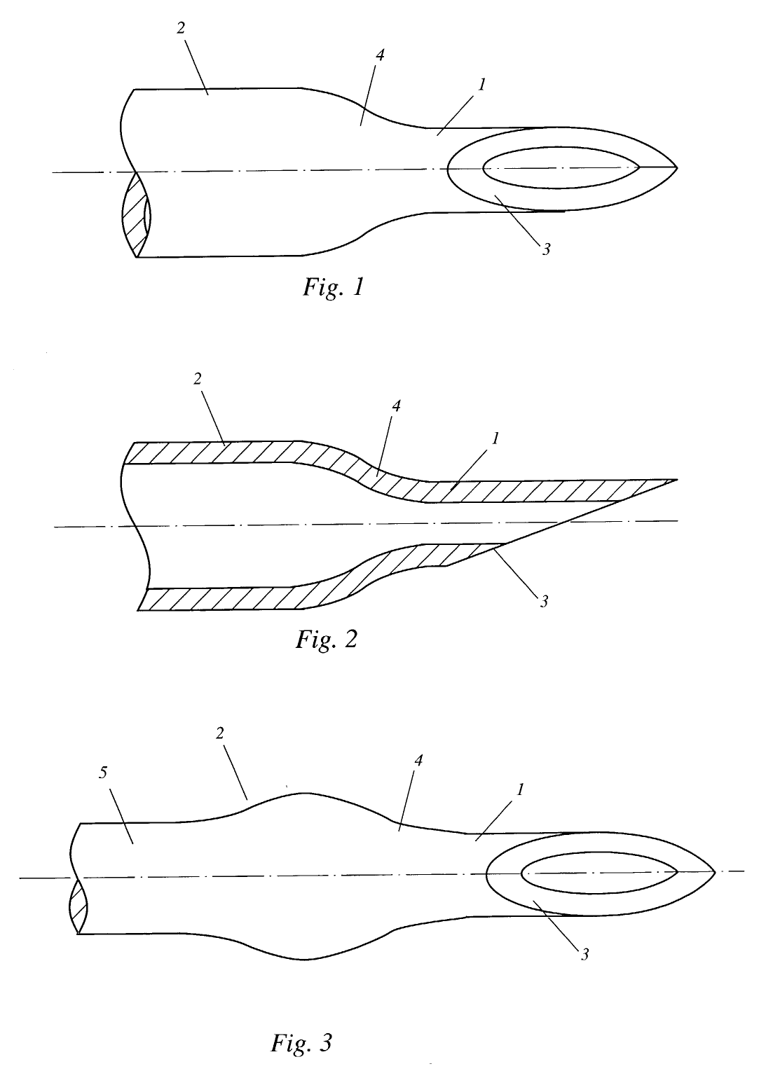

The outer diameter of the hollow needle illustrated in FIG. 1 and FIG. 2, which comprises a ground surface 3 at its tip, expands continuously from a region 1 near the tip but behind the ground surface 3 such that the outer diameter in the region 2 located further from the tip is approximately double that in region I. The rotationally symmetrical transition region 4 is conical and is rounded off at each end towards the regions 1, 2 which are respectively closer to the tip and further from the tip.

In use, the ground tip surface 3 of the puncture needle or cannula cuts through the skin, the intermediate tissue and the wall of the blood vessel. The wider part 2 of the hollow needle causes the puncture point to widen. Consequently, it is possible, in a single operational step, to gain those advantages that were only obtained with the additional employment of a dilator when using conventional puncture needles: Due to having a primary puncture of small diameter and then widening the puncture hole to the diameter required for introducing a correspondingly thick catheter, the traumatic injury to the blood vessel thereafter recedes relatively quickly back to the smaller diameter of the puncture. Thus, in contrast to the use of a uniformly thick needle having just one outer diameter, the time for healing the wound is limited to that required for a smaller hole. The healing process is thereby reduced and the patient is cared for as fully as possible.

FIG. 3 shows a further embodiment of the invention in which the second region 2 is bulb-shaped and is followed in proximal direction by a third section 5 whose outer diameter is approximately equal to the outer diameter of the first region. When using this hollow needle for puncturing the blood vessel, the puncture hole cut into the blood vessel is widened by passing the second region there through. Thereafter, the third region 5 arrives at the puncture hole which relieves the expansion stress to the vessel tissue. After having inserted a catheter guide wire into the hollow needle, the hollow needle is withdrawn while holding the guide wire stationary. When withdrawing the hollow needle, the bulb-shaped second region passes the puncture hole again to expand it once more to prepare it finally for the reception of a catheter which is channeled over the guide wire into the blood vessel.

It will be understood that the above description of the present invention is susceptible to various modifications, changes and adaptations.

What is claimed is:

1. A hollow needle for puncturing a blood vessel, and for introducing a wire for the placement of a catheter according to the Seldinger technique, said hollow needle comprising:
a body having a tip for insertion into said blood vessel thus creating a puncture hole in said blood vessel, said tip located at a distal end of said body;
a first region located adjacent said tip, said first region having a first outer diameter, and a second region located at a longitudinal distance further away from said tip than said first region, said second region having a second outer diameter greater than said first outer diameter of said first region;
wherein said second region is adapted to widen said puncture hole by being at least partially inserted into said blood vessel and has an outer diameter between approximately 1.3 mm and 5.4 mm.

2. A hollow needle in accordance with claim 1, wherein said body further includes a ground surface adjacent said distal tip.

3. A hollow needle in accordance with claim 1, wherein a ratio of said second outer diameter to said first outer diameter is between approximately 1.2 and 3.0.

4. A hollow needle in accordance with claim 1, wherein said first outer diameter is between approximately 0.4 mm and 1.5 mm.

5. A hollow needle in accordance with claim 1, wherein said first outer diameter is between approximately 0.7 mm and 1.8 mm.

6. A hollow needle in accordance with claim 1 wherein said body has a cross section expanding continuously in a transition region from said first outer diameter to said second outer diameter.

7. A hollow needle in accordance with claim 6, wherein said body has a conical shape in said transition region.

8. A hollow needle in accordance with claim 7, wherein said transition region has an opening angle lying between approximately 10° and 45°1′.

9. A hollow needle in accordance with claim 6, wherein said body further includes a ground surface located adjacent to said tip, said transition region located aft of said ground surface.

10. A hollow needle in accordance with claim 6, wherein said transition region between said first and the second outer diameters is rounded off.

11. A hollow needle in accordance with claim 6, wherein said first region and said second region and said transition region are formed as one piece.

12. A hollow needle in accordance with claim 1, wherein said body has a third region located at a longitudinal distance further away from said tip than said second region, said third region having a third outer diameter smaller than said second outer diameter of said second region.

13. A hollow needle in accordance with claim 12, wherein said third outer diameter is approximately equal to said first diameter and said second region having the shape of a bulb.

14. A method for creating a puncture hole in a blood vessel of a patient and widening said puncture hole for introducing a catheter into said blood vessel, comprising the steps of:
providing a hollow needle comprising a tip located at a distal end of said hollow needle, a first outer diameter located in a first region adjacent said tip and a second outer diameter located in a second region located at a longitudinal distance further away of said distal end than said first region, said second outer diameter being greater than said first outer diameter;
puncturing said blood vessel using said tip of said hollow needle in order to create said puncture hole;
introducing said first region and at least a part of said second region through said puncture hole into said blood vessel in order to widen said puncture hole, wherein, after completing said step of puncturing said blood vessel, said tip remains in said blood vessel until completion of said step of introducing said first region and said part of said second region.

15. A method for creating a puncture hole in a blood vessel of a patient and widening said puncture hole for introducing a catheter into said blood vessel, comprising the steps of:
providing a hollow needle comprising a tip located at a distal end of said hollow needle, a first outer diameter located in a first region adjacent said tip and a second outer diameter located in a second region located at a longitudinal distance further away of said distal end than said first region, said second outer diameter being greater than said first outer diameter;
puncturing said blood vessel through said tip of said hollow needle in order to create said puncture hole;
introducing said first region and at least a part of said second region through said puncture hole into said blood vessel in order to widen said puncture hole, wherein said steps of puncturing said blood vessel and introducing said first region and said part of said second region are performed by one substantially continuous movement without removing said tip before introducing said first region and said part of said second region.

16. A method for introducing a catheter comprising at least one lumen into a patient's blood vessel according to the Seldinger technique, said method comprising the steps of:
providing a hollow needle comprising a tip located at a distal end of said hollow needle, a first outer diameter located in a first region adjacent said tip and a second outer diameter located in a second region located at a longitudinal distance further away of said distal end than said first region, said second outer diameter being greater than said first outer diameter;
puncturing said blood vessel using said tip of said hollow needle in order to create said puncture hole;
dilating said puncture hole by introducing said first region and at least a part of said second region through said puncture hole into said blood vessel immediately after puncturing said blood vessel;
introducing a part of a guide wire into said blood vessel through said hollow needle after dilating said puncture hole;
removing said hollow needle completely from said blood vessel leaving said part of said guide wire in said blood vessel;
threading said catheter onto said guide wire; and
introducing said catheter along said guide wire through said dilated puncture hole into said blood vessel.
